(19) United States
(12) Patent Application Publication
(10) Pub. No.: US 2020/0239832 A1
(43) Pub. Date: Jul. 30, 2020

(54) TOLERANCE OF MICROBIAL CELLS AGAINST ORTHO-AMINOBENZOATE IN PRESENCE OF ALKALI IONS AT NEUTRAL PH

(71) Applicant: Covestro Deutschland AG, Leverkusen (DE)

(72) Inventors: Gernot JÄGER, Köln (DE); Wolf KLOECKNER, Köln (DE); Swantje BEHNKEN, Sacramento, CA (US); Simon KLAFFL, Düsseldorf (DE); Jamaleddine SASSI, Köln (DE)

(21) Appl. No.: 16/755,270

(22) PCT Filed: Oct. 12, 2018
(86) PCT No.: PCT/EP2018/077872

§ 371 Date: Apr. 10, 2020

(30) Foreign Application Priority Data
Oct. 12, 2017 (EP) 17196155.0

(51) Int. Cl. C12N 1/20; C12P 13/00

(52) U.S. Cl. CPC C12N 1/20; C12P 13/001

(57) ABSTRACT

The present invention relates to the production of o-aminobenzoic acid from fermentable substrates using microbial cells and alkali-containing bases during fermentation

The present invention relates to the production of o-aminobenzoic acid from fermentable substrates using microbial cells and alkali-containing bases during fermentation.

Currently, there is no renewable or biologically derived source of o-aminobenzoate or the corresponding acid commercially available. Current production methods of aniline rely on chemical synthesis from petroleum-derived raw-materials. Such petroleum-derived raw materials are not renewable as opposed to raw materials which are renewable, such as the renewable resource “biomass”. The chemical synthesis of aniline is a multi-step process. The several reaction steps involved in the production of aniline result in high production costs. Moreover, the conventional, i.e. chemical, synthesis of aniline is associated with hazardous intermediates, solvents, and waste products which can have substantial impacts on the environment. Non-specific side-reactions on the aromatic-ring result in the reduction of the product yield, thus further increasing the production costs. Petroleum-derived raw materials are influenced by cost fluctuations resulting from the global petroleum price.

o-aminobenzoate is a natural intermediate of the shikimate acid pathway and a precursor for the biosynthesis of the aromatic amino acid L-tryptophane. WO 2015/124687 discloses a concept of producing biologically-derived aniline in two process steps: (1) the fermentative production of o-aminobenzoate using recombinant bacteria and (2) the subsequent catalytic conversion of o-aminobenzoic acid into aniline. The recombinant bacteria used in said process belong to the family of Corynebacterium or Pseudomonas. Both bacteria produce o-aminobenzoate at a pH between 7 and 8.

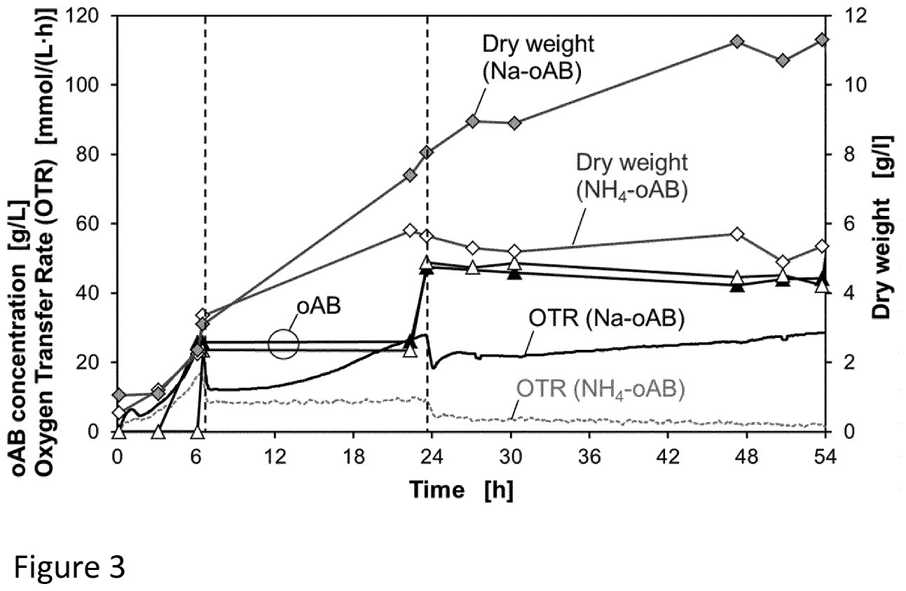

The following problem exists when producing o-aminobenzoate between pH 7 and pH 8: Due to the fermentative production of o-aminobenzoate which is an acid, a base such as NH₄OH, needs to be added in order to ensure a stable neutral pH. Thereby, a salt of e.g. NH₄⁺/o-aminobenzoate⁻ is produced. However, such o-aminobenzoate salts are toxic to microbial cells. According to FIG. 3, the metabolic activity of bacterial cells (see OTR) is limited when NH4⁺/o-aminobenzoate concentrations of more than 25 g/L o-aminobenzoate are reached and cell growth (see dry weight) stops at higher concentration (>50 g/L). This toxicity is not known for other products as glutamate or lysine.

This type of toxicity problem is typically solved by direct evolution of the applied microbial cells. First, microbial cells are exposed to increasing concentration of the toxic component (e.g. o-aminobenzoate) in repeated batch experiments or continuous fermentation trials. Thereby, the microbial cells evolve by random mutagenesis (which can be accelerated by adding mutagens) and the more resistant microbial cells survive. Secondly, the most resistant cells are isolated/selected and can be used for production.

However, many of the mechanisms underlying resistance in such microbial cells consume energy (Aindrila Mukhopadhyay. Trends in Microbiology, August 2015, Vol. 23, No. 8; Rau et al. Microb Cell Fact (2016) 15: 176; Warnecke T, Gill RT. Microbial Cell Factories. 2005; 4: 25). Thus, a certain proportion of the fermentable substrate is consumed for maintenance metabolism leading to decreased yields of o-aminobenzoate. For this reason, the high titers of o-aminobenzoate which are required for a reasonable space-time yield of the process concomitantly decrease product yield.

Although biotechnological production of o-aminobenzoate from renewable sources as a precursor for aniline production offers potential benefits, the above-described factor of toxicity diminishes the potential benefits of this process. Therefore, there is a need for alternative methods for increasing the resistance of microbial cells towards o-aminobenzoic acid.

This problem is solved by the embodiments defined in the claims and the description below.

In a first embodiment, the present invention relates to a method for cultivating a microbial cell in the presence of at least 30 g/l ortho-aminobenzoate (oAB) comprising the step of adding ions of alkali metals to the culture medium so that the molar ratio of alkali ions and oAB is in the range between 0.75 and 1.25.

The microbial cell is, preferably, a cell which is capable of biologically converting a fermentable substrate into oAB. The term “biologically converting” refers to the biochemical processes which transform one or more molecules of the fermentable substrate into one or more molecules oAB. These processes are predominantly mediated by enzymes expressed by the bacterial cell.

The term “o-aminobenzoic acid” (or oAB) as referred to in the present application relates to 2-aminobenzoic acid. This compound is also known as anthranilic acid. The person skilled in the art knows that an acid may be present in its protonated form as neutral substance or deprotonated as anion. In aqueous solution a part of the acid is protonated and a part is present as anion. The ratio between protonated acid and anion depends on the pH of the solution and the dissociation constant Kₐ of the acid in question. Unless indicated otherwise, the term “o-aminobenzoic acid” as used in this application always refers to both the protonated acid as well as to the corresponding anion.

The term “culture medium” is generally understood in the art. It refers to an aqueous solution which provides conditions which allow metabolic activity of the microbial cell. Said conditions are physico-chemical such as temperature, concentration of dissolved oxygen, ion strength and pH. They are also chemical and include the concentration of the different nutrients required by the microbial cell for its activity. The person skilled in the art can adapt these conditions to the needs of a particular microbial cell based on the common knowledge available for the particular microbial cell.

The microbial cell used in the present invention may be a naturally occurring strain, i.e. a microbial strain which is without any further human interaction, particularly without genetic manipulation capable of converting a fermentable substrate into oAB. However, in a preferred embodiment of the present invention it is a microbial cell which gained the aforementioned capability in the process of genetic manipulation or a microbial cell, where such methods were used to improve a pre-existing capability.

The term “genetic modification” within the meaning of the invention refers to changes in nucleic acid sequence of a given gene of a microbial host as compared to the wild-type sequence. Such a genetic modification can comprise deletions as well as insertions of one or more deoxyribonucleic acids. Such a genetic modification can comprise partial or complete deletions as well as insertions introduced by transformations into the genome of a microbial host. Such a genetic modification can produce a recombinant microbial host, wherein said genetic modification can comprise changes of at least one, two, three, four or more single nucleotides as compared to the wild type sequence of the respective microbial host. For example, a genetic modification can be a deletion or insertion of at least one, two, three, four or more single nucleotides or a transformation of at least one, two, three, four or more single nucleotides. A genetic modification according to the invention can have the effect of e.g. a reduced expression of the respective gene or of e.g. an enhanced expression of the respective gene.

The microbial cell is a prokaryotic cell or an eukaryotic cell. Preferably, the prokaryotic cell is a bacterial cell. Preferred bacterial cells belong to genera Corynebacterium, Mycobacterium, Bacillus, Pseudomonas, Escherichia, and Vibrio. More preferred are Corynebacterium glutamicum and Pseudomonas putida. Most preferred is Corynebacterium glutamicum ATCC 13032. Preferred eukaryotic cells belong to the order Saccharomycetales or the genus Aspergillus. More preferably, they belong to the species Ashbya gossypii, Pichia pastoris, Hansenula polymorpha, Yarrowia lipolytica, Zygosaccharomyces bailii, Kluyveromyces marxianus and Saccharomyces cerevisiae. Most preferably, the yeast is Saccharomyces cerevisiae.

It is particularly preferred that said microbial cell is characterized by a genetic modification of the trpD gene which prevents or decreases the expression of said gene and/or which leads to a gene product with decreased or without enzymatic activity. The person of average skill in the art can easily generate such microbial cells using conventional genetic methods.

Recombinant bacterial cells which are particularly suitable for the method of the present invention are disclosed in WO 2015/124687.

The term “cultivating” refers to the incubation of the microbial cell under conditions which facilitate metabolic activity. Such conditions are known to the person skilled in the art. Said conditions minimally encompass presence of the microbial cell in a culture medium suitable for growth of the cell at temperatures which allow cell proliferation, presence of a fermentable substrate and presence of oxygen. Preferably, said metabolic activity is oxygen consuming. More preferably, the metabolic activity is cell proliferation as measured by the increase of dry weight. Most preferably, the metabolic activity is the biological conversion of the fermentable substrate to oAB.

Preferably, the cultivation is performed in a culture medium having a pH between 6.0 and 8.0. Preferably, the pH is maintained in this ranged as described below in this application.

A fermentable substrate as understood by the present application is any organic compound or mixture of organic compounds which can be utilized by the microbial cell to produce o-aminobenzoic acid in the presence or absence of oxygen. Preferred fermentable substrates additionally serve as energy and carbon sources for the growth of the microbial cell. Preferred fermentable substrates are processed sugar beet, sugar cane, starch-containing plants and lignocellulose. Also preferred as fermentable substrate are glycerol and C1-compounds, preferably CO, and fermentable sugars. A preferred fermentable sugar is glucose.

Alkali ions suitable for the method of the present invention are the ions of all alkali metals. Preferred alkali metals are sodium, potassium and rubidium. A particularly preferred alkali metal is sodium. According to the invention mixtures of at least two different alkali ions may be used as well.

Ions of alkali metals are, preferably, added to the culture medium as constituents of a base comprising said ions. In the present invention, such a base is referred to as “alkali base”. More preferably, said alkali base is a hydroxide. Alternative alkali metal containing bases include carbonates or phosphates such as disodium phosphate. It is to be understood that the term “alkali base” also refers to mixtures of at least two different alkali bases.

In a further embodiment, the present invention relates to a method for producing oAB comprising the steps of
a) incubating a microbial cell in the presence of said fermentable substrate and under conditions suitable for the biological conversion of said fermentable substrate into oAB; and
b) adding an alkali base to the fermentation broth as buffer substance.

All definitions given above also apply to this embodiment.

The person skilled in the art is able to select incubation conditions which are suitable for the biological conversion of a fermentable substrate into oAB based on the known properties and culture requirements of the microbial cell. Whether the culture conditions are suitable for oAB production may easily be determined by measuring the concentration of oAB in the culture medium. An increase of the oAB concentration over time indicates that the culture conditions fulfill the requirements.

A “fermentable substrate” is any carbon compound which may be converted to oAB by the microbial cell. Typically, the fermentable substrate will additionally feed the maintenance and/or growth metabolism of the microbial cell. However, in special cases it may be necessary to complement the fermentable substrate by additional substrates which feed the maintenance and/or growth metabolism of the microbial cell. Preferred fermentable substrates are selected from the group consisting of C-5 monosaccharides, C-6 monosaccharides, disaccharides, and tri-saccharides. The C-5 monosaccharides are, preferably, xylose and arabinose. The C-6 monosaccharides are, preferably, glucose, fructose or mannose. The disaccharide is, preferably, saccharose. The trisaccharide is, preferably, kestose.

It is preferred that the dry weight of the microorganisms at least doubles during the course of the incubation. More preferably, the dry weight at the end of the incubation reaches at least 6 g/l.

Since oAB is an acid, the addition of a base is necessary to keep the pH of the culture medium stable. However, in addition to its effect on pH which can be mitigated by addition of a base, above certain concentrations oAB itself is toxic for microorganisms. In the study underlying the present invention it has been surprisingly found that the same strain of bacteria tolerates higher concentrations of oAB if the base used for maintaining a stable pH contains an alkali ion as cation.

The total amount of all bases added in method step b) depends on the amount of oAB produced up the time, where the base is added. It must be sufficient to prevent excessive drop of pH, while not leading to an increase of pH above the range which is tolerated by the microorganism in question. Preferably, by addition of the base the pH is maintained in a range, where the metabolic activity of the microorganism in question is at least 60%, more preferably at least 80%, of the activity at optimal pH. All definitions for “metabolic activity” given above also apply here. Hence, the addition of the base with regard to time and amount is preferably based on the measurement of pH in the culture medium as commonly practiced in industrial biotechnology. The addition may take place in a continuous fashion as a steady stream. It may also be performed by adding discrete dosages of the bases at different points in time.

From the line of argument set forth above it follows that method step b) is performed in parallel with method step a), i.e. the sodium-containing base is added while the incubation of the microorganism takes place.

In the presence of an alkali base, particularly a sodium containing base, a microbial cell as defined above tolerates at least 200%, more preferably at least 170%, even more preferably at least 150% and most preferably at least 130% of the oAB concentration which is tolerated by the same microbial cell in the absence of the alkali base under otherwise identical culture conditions.

Preferably, the minimum concentration of oAB tolerated is 30 g/l, more preferably 40 g/l and most preferably 50 g/l.

According to the invention it is not required that the base added in method step b) exclusively consists of an alkali base. It is also envisaged by the present invention that mixtures of alkali bases and other bases are used in method step b). “Other bases” are, for example gaseous ammonium, ammonium hydroxide, calcium hydroxide or calcium carbonate. However, it is preferred that a large proportion of the base added in method step b) is a sodium containing base. In this context it is important to note that other bases, particularly those containing nitrogen, may be consumed by the microbial cell. Therefore it is preferred to define the ratio of alkali base and “other bases” not by the amount added but by the molar ratio of alkali and other bases actually present in the culture medium at a given point in time. Preferably, the addition of alkali and other bases in method step b) is performed in such a way that the molar amount of the alkali base makes up at least 30 mol-%, more preferably at least 50 mol-%, even more preferably at least 75 mol-% and most preferably at least 90 mol-%. It is preferred that these limits are kept over the entire incubation time. However, for practical reasons it is acceptable that the amount of the alkali base drops below the values defined above as long as the above-defined molar ratios are kept during at least 90% of the duration of the total incubation time.

The aforementioned percentages refer to the amount of cations. In the most preferred embodiment, the base added in method step b) does not contain cations other than sodium within the degree of purity of reagents typically employed in large scale fermentations. A typical sodium containing base is NaOH which is a side product of the chlor-alkali process. This base has a concentration of approximately 50 weight-% NaOH.

In a particularly preferred embodiment of the present invention, the amount of ammonium defined as the sum of the concentrations of NH₃ and NH₄⁺ in the culture medium does not exceed 300 mM, preferably 200 mM, more preferably 100 mM and even more preferably 50 mM.

A microbial cell “tolerates” a given amount of oAB if its metabolic activity as defined above in this application does not decrease by more than 50%, more preferably not more than 25% below the activity shown in the absence of oAB. It is to be understood that any sudden increase of oAB concentration causes a transient decrease of the metabolic activity of the microbial cell (see examples). During the process of oAB production this effect is unlikely to be encountered because oAB concentrations increase gradually so that the microbial cell has time to adapt. However, addition of oAB for testing purposes may have this effect. Therefore it is preferred that the metabolic activity of a microbial cell is measured at least two hours after any sharp increase of oAB concentration, e.g. caused by the addition of oAB to the culture medium. Otherwise the true and lasting effect of oAB on the activity may be overestimated for the given conditions.

If oAB is produced by the microbial cell under the conditions in questions, it accumulates in the microbial cells and/or the culture medium. The person skilled in the art is well aware of a multitude of methods suitable for recovering the desired product from the cells or the culture medium. Preferred methods are disclosed in WO 2015/124687.

Advantageously, the use of alkali bases allows the growing of a given microbial strain in the presence of higher concentrations of oAB as compared to the use of other bases. Thus, an increased oAB tolerance could be reached without development of novel strains which is a time consuming process. Moreover, many mechanisms underlying oAB resistance of specifically engineered strains consume energy so the less fermentable substrate is available for the actual conversion into oAB leading to decreased substrate yield.

In another embodiment, the present invention relates to the use of an alkali base as buffer substance during the biological conversion of a fermentable substrate into oAB by a microbial cell.

In yet another embodiment, the present invention relates to the use of ions of alkali metals in order to increase the tolerance of microbial cells towards oAB. The alkali metal ion is preferably a sodium or potassium ion.

In yet another embodiment, the present invention relates to the use of an alkali base in order to increase the tolerance of microbial cells towards oAB.

The following examples are only intended to illustrate the invention. They shall not limit the scope of the claims in any way.

### EXAMPLES

The figures show:

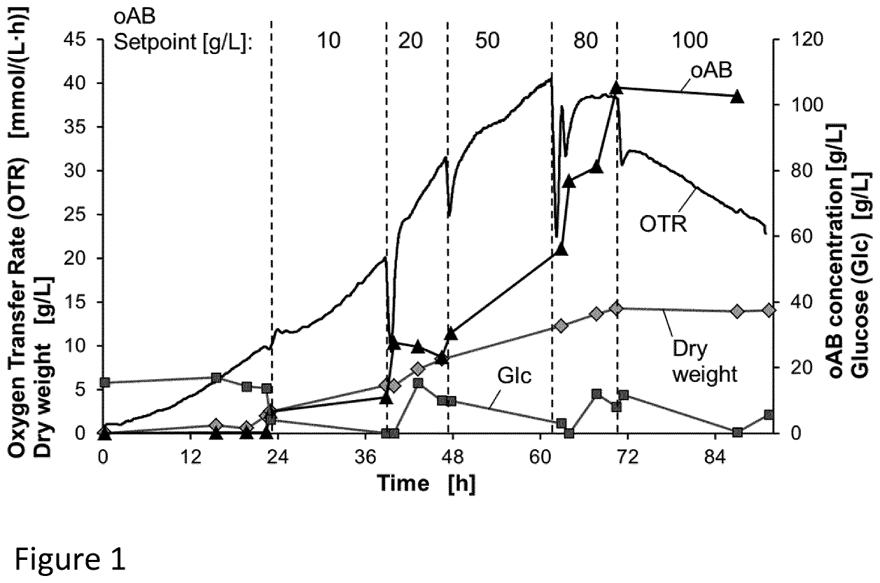

FIG. 1: Evaluation of the maximum tolerable oAB concentration during batch cultivation in a 1 L lab-scale bioreactor with a modified C. glutamicum production strain. Addition of 20 mL of a 500 g/L oAB stock solution (oAB dissolved with NaOH at pH 7) after 23 h and 39 h, 48 h (two times), 64 h (two times) and 71 h (two times) and addition of glucose solution after 40 h, 48 h, 63 h, 65 and 71.5 h to prevent a glucose limitation. Initial cultivation volume VL=1 L, temperature 30° C., pH=7 controlled by adding NH₄OH (10 w % NH₃) during cultivation, gassing rate with air=0.2 L/min, pO₂ controlled at 30% air saturation by adjusting the stirrer speed between 200 and 1200 rpm.

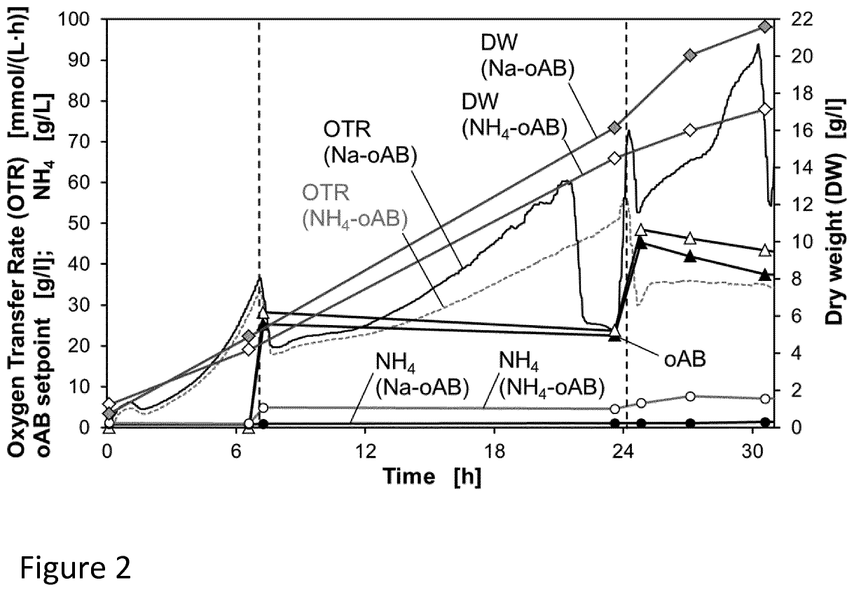

FIG. 2: Influence of Na-oAB and NH₄-oAB addition on oAB resistance during batch cultivation in 1 L scale with C. glutamicum ATCC 13032. Filled symbols: addition of 40 mL of 500 a oAB stock solution (oAB dissolved with NaOH at pH 7) after 7 h and 24 h. Open symbols: addition of 40 mL of 500 g/L oAB stock solution (oAB dissolved with NH4OH at pH 7) after 7 h and 24 h. Both bioreactors: addition of 36 g/L glucose (added as stock solution) after 7.5 h and 24.5 h to prevent a glucose limitation. Initial cultivation volume VL=1 L, temperature 30° C., pH=7 controlled with NH₄OH (10 w % NH₃), gassing rate with air=0.2 L/min, pO₂ controlled at 30% air saturation by adjusting the stirrer speed between 200 and 1200 rpm.

FIG. 3: Influence of Na-oAB and NH4-oAB addition on oAB resistance during batch cultivation in 1 L scale with C. glutamicum ATCC 13032. Filled symbols: addition of 40 mL of 500 g/L oAB stock solution (oAB dissolved with NaOH at pH 7) after 6.4 h and 23.6 h. Open symbols: addition of 40 mL of 500 g/L oAB stock solution (oAB dissolved with NH₄OH at pH 7) after 6.4 h and 23.6 h. Initial cultivation volume VL=1 L, temperature 30° C., pH=7 controlled with NH₄OH (10 w % NH₃), gassing rate with air=0.2 L/min, pO₂ controlled at 30% air saturation by adjusting the stirrer speed between 200 and 1200 rpm.

### EXAMPLE 1

### Evaluation of the Maximum Tolerable oAB Concentration of a Modified C. glutamicum Production Strain

The maximum tolerable oAB concentration in the presence of sodium ions in the medium was tested with a modified C. glutamicum production strain. This strain was derived from Corynebacterium glutamicum ATCC 13032 by evolutionary engineering in order to increase its resistance against oAB.

This strain was then genetically modified to make it capable of oAB-production. The incorporated modifications have the effect of reduced expression of the trpD gene, encoding anthranilate phosphoribosyl transferase, knock out of gene ppc, encoding PEP Carboxylase, constitutive overexpression of heterologous aroG^{D146N} and trpEG⁵⁴⁰ genes from E. coli, encoding feedback resistant DAHP synthase and anthranilate synthase, respectively, constitutive overexpression of the gene aroL from E. coli, encoding shikimate kinase.

One bioreactor with a nominal volume of 1 L was filled with sterile cultivation medium including an initial amount of 20 g/L glucose, 5 g/L (NH₄)₂SO₄, 1 g/L KH₂PO₄, 1 g/LK₂HPO₄, 0.25 g/L MgSO₄.7 H₂O, 0.01 g/L CaCl₂.2 H₂O, 2 mg/L biotin (vitamin B7), 0.03 g/L protocatechuic acid (3,4-Dihydroxybenzoic acid), 0.01 g/L MnSO₄.H₂O. 0.01 g/L FeSO₄.7H₂O, 1 mg/L ZnSO₄.7H₂O, 0.2 mg/L CuSO₄.5H₂O and 0.02 mg/L NiCl₂.6H₂O.

The preculture medium for the cultivation in shake flasks contained additionally 42 g/L MOPS buffer, 3.7 g/L brain heart infusion broth, 5 g/L urea (CH₄N₂O) and 20 g/L (NH₄)₂SO₄ (instead of 5 g/L). The preculture was cultivated in 300 mL shake flasks with a liquid volume of 25 mL at a temperatur of 28° C. and a shaking frequency of 180 rpm until OD₆₀₀>20 was reached.

The cultivation was performed in a lab scale bioreactor with an initial cultivation volume of 1 L. Temperature was controlled at 30° C. and pH was kept constant at pH=7 by adding aqueous NH₄OH solution (10 w % NH₃) during the fermentation. The gassing rate was adjusted to 0.2 L/min air and the dissolved oxygen tension was controlled at 30% air saturation by controlling the stirrer speed between 200 rpm and 1200 rpm. Results of the cultivation are shown in FIG. 1. The oAB concentration was increased stepwise by adding 20 mL of a 500 a oAB stock solution (oAB dissolved with NaOH at pH 7) after 23 h, 39 h, 48 h (two times), 64 h (two times) and 71 h (two times). Glucose was added as stock solution after 40 h, 48 h, 63 h, 65 and 71.5 h to prevent a glucose limitation. An increase in biomass concentration was observed even at an oAB concentration of 80 a as shown in FIG. 1. Increasing the oAB concentration from 80 g/L to 100 g/L after 71 h resulted in a decrease of the metabolic activity (indicated by the declining OTR signal) and no further increase in biomass was observed at that point. With this experiment it was demonstrated that growth of the C. glutamicum production strain in the presence of 80 g/L oAB can be achieved by adding oAB as sodium salt to the bioreactor.

### EXAMPLE 2

### Comparison of the Metabolic Activity after NH₄-oAB and Na-oAB Addition during the Cultivation of C. glutamicum ATCC 13032

A C. glutamicum strain derived from ATCC 13032 was used to compare the metabolic activity after NH₄-oAB and Na-oAB addition during the cultivation. This strain was derived from Corynebacterium glutamicum ATCC 13032 by evolutionary engineering in order to increase its resistance against oAB.

It was not further genetically modified. Thus it was not capable of oAB-production. For this purpose, two 1 L bioreactors were filled with sterile cultivation medium including the following initial concentrations: 20 g/L glucose, 5 g/L (NH₄)₂SO₄, 1 g/L KH₂PO₄, 1 g/L K₂HPO₄, 0.25 g/L M_{g}SO₄.7 H₂O, 0.01 g/L CaCl₂.2 H₂O, 2 mg/L biotin (vitamin B7), 0.03 g/L protocatechuic acid (3,4-Dihydroxybenzoic acid), 0.01 g/L MnSO₄.H₂O, 0.01 g/L FeSO₄.7H₂O, 1 mg/L ZnSO₄.7H₂O, 0.2 mg/L CuSO₄.5H₂O and 0.02 mg/L NiCl₂.6H₂O.

The preculture medium for the cultivation in shake flasks contained additionally 42 g/L MOPS buffer, 3.7 g/L brain heart infusion broth, 5 g/L urea (CH₄N₂O) and 20 a (NH₄)₂SO₄ (instead of 5 g/L). The preculture was cultivated in 300 mL shake flasks with a liquid volume of 25 mL at a temperatur of 28° C. and a shaking frequency of 180 rpm until OD₆₀₀>20 was reached.

Results for dry biomass, oAB and NH₄ concentrations and the Oxygen Transfer Rate (OTR) signals are shown in FIG. 2. After addition of 40 mL of a 500 g/L oAB stock solution (oAB dissolved with NaOH at pH 7) at a cultivation time of 7 h and 24 h to reactor 1, the biomass concentration and OTR signal continued to increase (filled symbols in FIG. 2). In contrast to that, the addition of NH₄-oAB (added by injection of 40 mL of a 500 g/L oAB stock solution containing oAB dissolved with NH₄OH at pH 7) to reactor 2 after 24 h resulted in a constant OTR signal and a reduced biomass accumulation (open symbols in Fehler! Verweisquelle konnte nicht gefunden werden. 2). This experiment shows that the replacement of NH₄⁺ by Na⁺ as counter ion for oAB increases the tolerance of the C. glutamicum ATCC 13032 towards oAB.

### EXAMPLE 3

### Comparison of the Metabolic Activity after NH₄-oAB and Na-oAB Addition during the Cultivation of C. glutamicum ATCC 13032

The influence of NH₄ was tested with C. glutamicum ATCC 13032 (without further genetic modifications, not modified by evolutionary engineering) using two 1 L bioreactors filled with sterile cultivation medium including the following initial concentrations: 20 a glucose, 5 g/L (NH₄)₂SO₄, 1 g/L KH₂PO₄, 1 g/L K₂HPO₄, 0.25 g/L MgSO₄.7 H₂O, 0.01 g/L CaCl₂.2 H₂O, 2 mg/L biotin (vitamin B7), 0.03 g/L protocatechuic acid (3,4-Dihydroxybenzoic acid), 0.01 g/L MnSO₄.H₂O, 0.01 g/L FeSO₄.7H₂O, 1 mg/L ZnSO₄.7H₂O, 0.2 mg/L CuSO₄.5H₂O and 0.02 mg/L NiCl₂.6H₂O.

The preculture medium for the cultivation in shake flasks contained additionally 42 g/L MOPS buffer, 3.7 g/L brain heart infusion broth, 5 g/L urea (CH₄N₂O) and 20 g/L (NH₄)₂SO₄ (instead of 5 g/L). The preculture was cultivated in 300 mL shake flasks with a liquid volume of 25 mL at a temperatur of 28° C. and a shaking frequency of 180 rpm until OD₆₀₀>20 was reached.

During the fermentation the temperature was controlled at 30° C. and pH was kept constant at pH=7 by adding aqueous NH₄OH solution (10 w % NH₃). The gassing rate was adjusted to 0.2 L/min air and the dissolved oxygen tension was controlled at 30% air saturation by adjusting the stirrer speed between 200 rpm and 1200 rpm.

Results for dry biomass and oAB concentrations and the related signals for the Oxygen Transfer Rate (OTR) are shown in FIG. 3. 40 mL of a 500 g/L oAB stock solution (oAB dissolved with NaOH at pH 7) was added to reactor 1 after 6.4 h and 23.6 h (filled symbols) and 40 mL of a 500 g/L oAB stock solution (oAB dissolved with NH₄OH at pH 7) was added after 6.4 h and 23.6 h to reactor 2 (open symbols)

As shown in FIG. 3, the biomass accumulation continued after Na-oAB was added to reactor 1 (filled symbols in FIG. 3). In contrast to that, the addition of NH₄-oAB to reactor 2 caused a growth inhibition after 24 h (open symbols in FIG. 3). From this it follows that the toxicity of oAB on C. glutamicum ATCC 13032 in presence of high Na+ concentrations is reduced compared to the toxicity in presence of high NH₄⁺ concentrations.

The effects described above can be generalized at least to the genus Corynebacterium. A strain which underwent evolutionary engineering, a method which changes the genetic material of an organism in a random fashion, shows the same behavior as the strain used in example 3. Thus, the effect seems to be based on some rather fundamental functions of the cell which are not easily altered.

1.-14. (canceled)

15. A method for cultivating a microbial cell in the presence of at least 30 g/l ortho-aminobenzoate (oAB) comprising the step of adding ions of alkali metals to the culture medium so that the molar ratio of alkali metal ions and oAB is in the range between 0.75 and 1.25.

16. The method of claim 15, wherein alkali metal ions are sodium or potassium ions.

17. The method of claim 15, wherein the microbial cell is a bacterial cell belonging to the genus Corynebacterium.

18. A method for producing oAB, wherein oAB-concentrations of at least 30 g/l are reached comprising the steps of
a) incubating a microbial cell capable of producing oAB from a fermentable substrate in the presence of said fermentable substrate and under conditions suitable for the biological conversion of said fermentable substrate into oAB; and
b) adding an alkali base to the fermentation broth as buffer substance.

19. The method of claim 18, wherein the microbial cell is a bacterial cell or a yeast cell.

20. The method of claim 19, wherein the bacterial cell belongs to the genus Corynebacterium.

21. The method of claim 18, wherein the concentration of ammonia in the fermentation broth does not exceed 200 mM.

22. The method of claim 18, wherein microbial biomass reaches at least 6 g/l dry weight.

23. The method of claim 18, wherein the alkali base is sodium hydroxide or potassium hydroxide.

24. A method comprising utilizing an alkali base as buffer substance during the biological conversion of a fermentable substrate into oAB by a microbial cell capable of said conversion.

25. A method comprising utilizing ions of alkali metals to increase the tolerance of microbial cells towards oAB.

26. A method comprising utilizing an alkali base to increase the tolerance of microbial cells towards oAB.

27. The method of claim 23, wherein the alkali metal is sodium or potassium.

28. The method of claim 24, wherein the microbial cell is a bacterial belonging to the genus Corynebacterium.
